(19) United States
(12) Patent Application Publication
(10) Pub. No.: US 2015/0284788 A1
(43) Pub. Date: Oct. 8, 2015

(54) Sequencing Performance With Additives

(71) Applicant: QIAGEN GmbH, Hilden (DE)

(72) Inventors: Nan Fang, Neuss (DE); Dirk Loeffert, Dusseldorf (DE); Varuna Mohan, Allston, MA (US)

(73) Assignee: QIAGEN GmbH

(21) Appl. No.: 14/679,192

(22) Filed: Apr. 6, 2015

Related U.S. Application Data
(60) Provisional application No. 61/976,028, filed on Apr. 7, 2014.

(51) Int. Cl. C12Q 1/68

(52) U.S. Cl. CPC C12Q 1/6869

(57) ABSTRACT

Methods are described which utilize additives that ease secondary structure of DNA to improve sequencing performance. In one embodiment, said additive is betaine. The addition of betaine leads to decreased error rates.

### FIELD OF INVENTION

The present invention relates generally to nucleic acid sequencing. Methods are described which utilize additives that ease secondary structure of DNA to improve sequencing performance.

### BACKGROUND OF THE INVENTION

Many of the next-generation sequencing technologies use a form of sequencing by synthesis (SBS), wherein specially designed nucleotides and DNA polymerases are used to read the sequence of chip-bound, single-stranded DNA templates in a controlled manner. To attain high throughput, many millions of such template spots are arrayed across a sequencing chip and their sequence is independently read out and recorded.

There is a continued need for methods and compositions for increasing the fidelity of sequencing nucleic acid sequences.

### SUMMARY OF THE INVENTION

The present invention relates generally to nucleic acid sequencing. Methods are described which utilize additives that ease secondary structure of DNA to improve sequencing performance. In one embodiment, the present invention contemplates a method for sequencing a nucleic acid by detecting the identity of a nucleotide analogue incorporated into primer extension strand in a polymerase reaction, comprising: a) providing i) an additive that eases secondary structure of DNA; ii) sequencing primer; (iii) single-stranded template attached to a solid surface; (iv) polymerase and v) a mixture of different nucleotide analogues, wherein each different nucleotide analogue comprises a base selected from the group consisting of adenine, guanine, cytosine, thymine, and uracil, and their analogues; and a unique label attached through a cleavable linker to the base or to an analogue of the base; a deoxyribose; and a cleavable chemical group at the 3′-position of the deoxyribose; b) hybridizing the sequencing primer to the template so as to create a primer-template complex; b) adding said additive to said mixture of different nucleotide analogues so as to create an extension solution; d) exposing said primer-template complex to said extension solution under conditions such that the sequencing primer is extended by incorporating a first nucleotide analogue therein with said polymerase in a polymerase reaction so as to create a primer extension strand, wherein the incorporated nucleotide analogue terminates the polymerase reaction; and e) detecting the unique label attached to the nucleotide analogue that has been incorporated into the growing strand of DNA, so as to thereby identify the incorporated nucleotide analogue. In one embodiment, the method further comprises f) cleaving the cleavable linker between the nucleotide analogue that was incorporated into primer extension strand and the unique label; and cleaving the cleavable chemical group at the 3′-position of the deoxyribose to leave an --OH group. In one embodiment, the method further comprises g) extending the sequencing primer by incorporating a second nucleotide analogue therein. In one embodiment, the method further comprises h) detecting the unique label attached to said second nucleotide analogue so as to thereby identity of the second incorporated nucleotide. In one embodiment, said additive is betaine.

In one embodiment, the present invention contemplates a method for sequencing a nucleic acid by detecting the identity of a nucleotide analogue incorporated into primer extension strand in a polymerase reaction, comprising: a) providing i) sequencing primer, said sequencing primer hybridized to (ii) single-stranded template attached to a solid surface; (iii) polymerase and iv) an extension solution comprising an additive that eases secondary structure of DNA and one or more different nucleotide analogues, wherein each different nucleotide analogue comprises a base selected from the group consisting of adenine, guanine, cytosine, thymine, and uracil, and their analogues; and a unique label attached through a cleavable linker to the base or to an analogue of the base; a deoxyribose; and a cleavable chemical group at the 3′-position of the deoxyribose; b) exposing said primer hybridized to said template to said extension solution and said polymerase under conditions such that the sequencing primer is extended by incorporating a first nucleotide analogue therein with said polymerase in a polymerase reaction so as to create a primer extension strand, wherein the incorporated nucleotide analogue terminates the polymerase reaction; and c) detecting the unique label attached to the nucleotide analogue that has been incorporated into the growing strand of DNA, so as to thereby identify the incorporated nucleotide analogue. In one embodiment, the method further comprises d) cleaving the cleavable linker between the nucleotide analogue that was incorporated into primer extension strand and the unique label; and cleaving the cleavable chemical group at the 3′-position of the deoxyribose to leave an —OH group. In one embodiment, the method further comprises e) extending the sequencing primer by incorporating a second nucleotide analogue therein. In one embodiment, the method further comprises f) detecting the unique label attached to said second nucleotide analogue so as to thereby identity of the second incorporated nucleotide. In one embodiment, said additive is betaine.

### BRIEF DESCRPTION OF THE DRAWINGS

FIG. 1 is a bar graph showing that the addition of betaine leads to increased mapped and perfect reads in sequencing.

FIG. 2 is a bar graph showing that the addition of betaine leads to decreased error rate in sequencing.

FIG. 3 shows one embodiment of a general workflow used in next generation sequencing approaches. DNA is fragmented and modified with adapters, prior to amplification in an emulsion. The emulsion is broken and the amplified (typically clonally amplified) template is sequenced.

### DEFINITIONS

An additive that eases secondary structure of DNA is one that reduces the formation of secondary structures. GC-rich DNA is often problematic due to stable secondary structures in the DNA.

### DESCRIPTION OF THE INVENTION

The present invention relates generally to nucleic acid sequencing. Methods are described which utilize additives that ease secondary structure of DNA to improve sequencing performance.

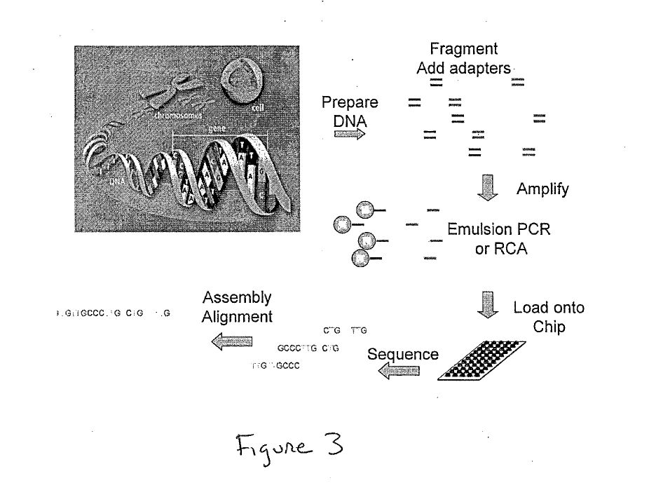

The QIAGEN GeneReader platform is a next generation sequencing (NGS) platform utilizing proprietary modified nucleotides whose 3′ OH groups are reversely terminated by a small moiety to perform sequencing-by-synthesis (SBS) in a massively parallel manner. Briefly, the sequencing templates are first clonally amplified on a solid surface (such as beads) to generate hundreds of thousands of identical copies for each individual sequencing template (see FIG. 3), denaturized to generate single-stranded sequencing templates, hybridized with sequencing primer, and then immobilized on the flow cell. The immobilized sequencing templates are then subjected to a nucleotide incorporation reaction in a reaction mix that includes modified nucleotides with a cleavable 3′ blocking group that enables the incorporation and specific fluorescent labels that enables detection of only one specific nucleotide onto each sequencing template in each cycle; a thermostable polymerase that works with such modified nucleotides, and suitable reaction buffer. See U.S. Pat. Nos. 6,664,079, and 8,612,161 and 8,623,598, hereby all incorporated by reference.

The sequencing performance depends strongly on the effectiveness and accuracy of the nucleotide incorporation step. One critical factor that can affect nucleotide incorporation efficiency is the secondary structure of the single-stranded sequencing templates, since the nucleotide incorporation reaction on GeneReader is performed as an isothermal reaction at 65° C. If the sequencing template forms complex secondary structure at this temperature, nucleotide incorporation could be potentially inhibited or completely blocked, causing incomplete nucleotide incorporation in certain cycles and therefore higher sequencing errors.

In one embodiment, the present invention contemplates a method that uses additives such as betaine that can ease secondary structure of DNA to improve sequencing performance,

### DESCRIPTION OF PREFERRED EMBODIMENTS

The present invention contemplates a novel method to improve SBS performance by optimizing sequencing chemistry. Specifically, the method is related to the use of additives in the nucleotide incorporation mixes to ease secondary structure of single-stranded nucleic acids and facilitate incorporation of the nucleotides. We show (see Experimental section below) that adding betaine, an isostabilizing agent, to the sequencing mixes can significantly reduce sequencing errors on GeneReader. It is not intended that the present invention be limited to the form of the betaine used, e.g. betaine mono-hydrate, betaine HCl, etc. are contemplated. It is also not intended that the present invention be limited to the concentration of betaine used. A wide range of final concentrations of 0.1M to 2.0M are contemplated.

It is not intended that the present invention be limited to a particular additive that eases secondary structure of DNA. A number of additives that can ease secondary structure of DNA, such as DMSO, glycerol, and single-strand DNA binding proteins, would also have similar benefits and are contemplated to be within under the scope of the invention as well as broad concentration ranges of the additives. Furthermore, we expect the combination of more than one such additive could potentially have additional benefits than using only one additive; and such combination is also under the scope of this invention.

It is also not intended that the present invention be limited to a particular sequencing platform. It is contemplated that the same or similar method could improve the performance of other SBS platforms in general (i.e. any sequencing-by-synthesis methods that operate under similar conditions), as well as specific SBS platforms, such as HiSeq and miSeq platforms from Illumina; Roche 454; Ion Torrent PGM and Proton platforms; and PacificBio platform.

### EXPERIMENTAL

We used betaine in the nucleotide mix as the additive to ease secondary structure. Betaine ((N,N,Ntrimethylglycine) is an isostabilizing agent that eases complex secondary structure by changing melting characteristics of DNA.

Briefly, 0.075 pg/μl PhiX library with the GeneReader—specific adaptors (X adaptor and B adaptor) was mixed with 2× GeneReader emulsion PCR Mix (1Xfinal concentration, containing polymerase, dNTPs, and buffer), Dynal MyOne C1 Streptavidin Beads (LIFE Technologies) conjugated with. M13 primer that has 5′ bisbiotin modification, PCR Primer B2 (6 μM final concentration) and 0.05 μM of bisbiotin modified forward primer to make up the aqueous phase of emulsion PCR. The aqueous phase and the proprietary QIAGEN oil phase, MB5, were mixed on the GeneRead QiaCube (Prototype) to generate water:oil emulsions. The emulsions were transferred to a 96-well PCR plate and subjected to PCR cycling with the following program:

### Initial Denaturation:

2 min@94 C

### Followed By

60 cycles of;
15 sec@94 C
15 sec@57 C
1 min 15 sec@72 C
Final Extension: 2 min@72 C

Following PCR, the emulsions were broken and washed on the GeneRead QiaCube Prototype with automated protocol. The beads were then washed twice in 1 ml PBST (0.05% Tween), denatured with NaOH/Tween solution (0.2N NaOH, 0.1% Tween) for 5 minutes at room temperature to generate single-stranded sequencing templates on the bead surface, and hybridized with GeneReader sequencing primer Seq46.

The emulsion PCR beads were then crosslinked to two different flow cells and sequenced on the GeneReader for 101 cycles. One flow cell was subjected in the sequencing reaction with standard GeneReader sequencing chemistry (Chem V4.0); the other flow cell was subjected in a sequencing reaction with GeneReader sequencing chemistry where the nucleotide incorporation mix also contains betaine at 0.5M final concentration.

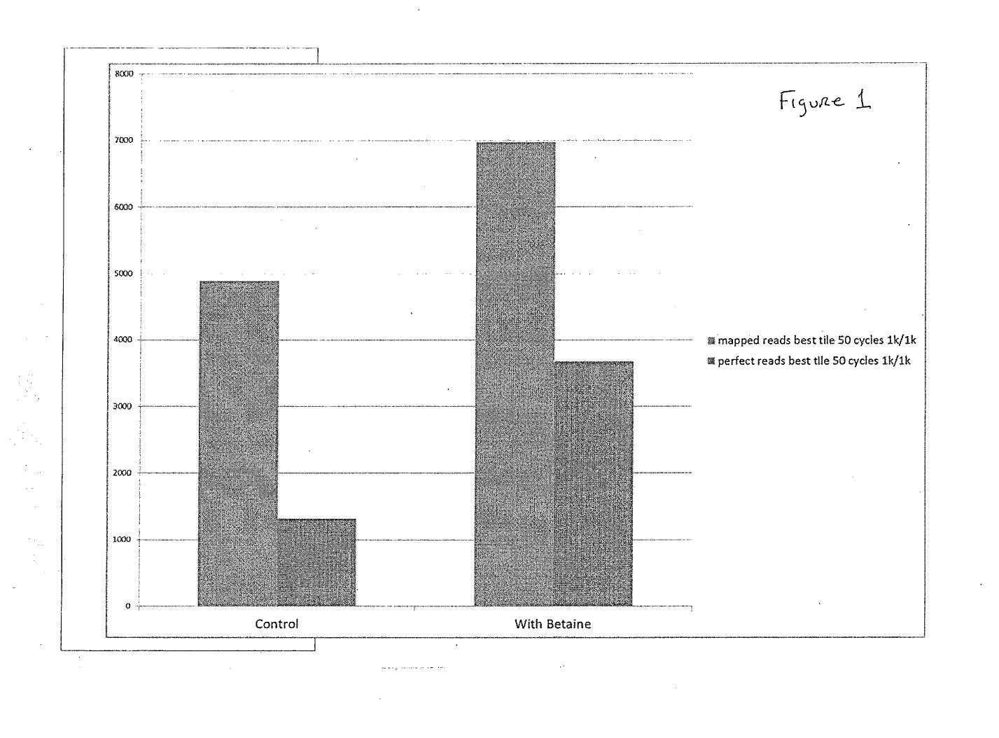

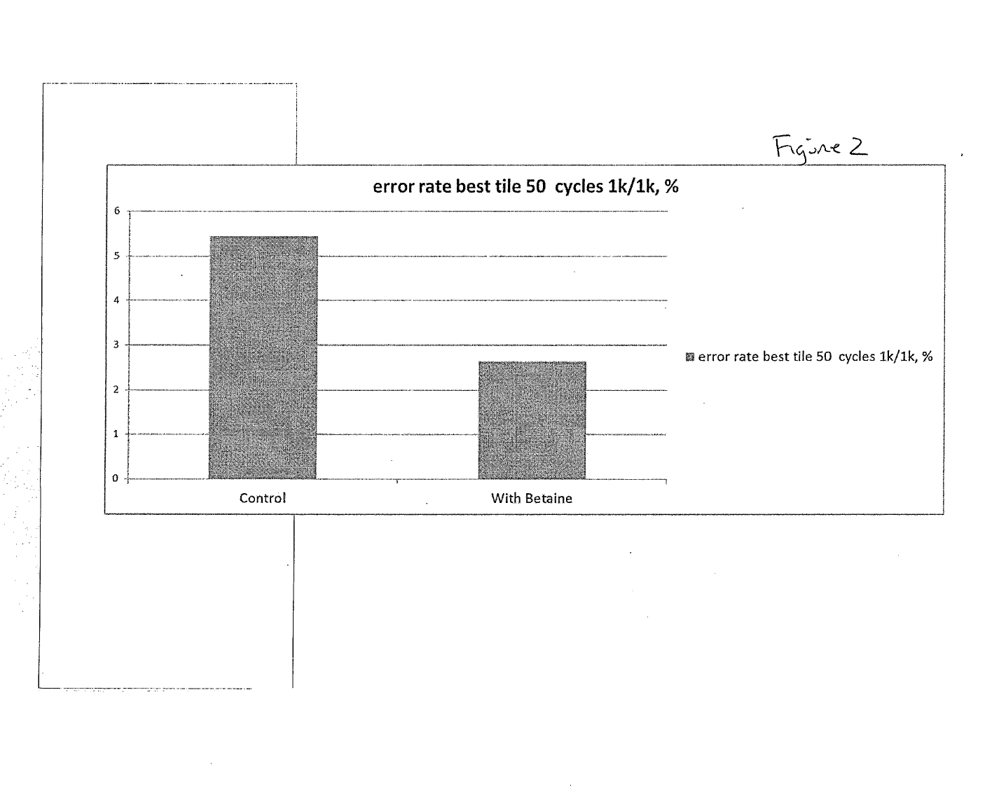

The beads were analyzed with IBS Analyze software (QIAGEN) for mapped reads (reads mapped to PhiX genome with less than 3 errors in the first 28 nucleotides), perfect reads (reads perfectly mapped to the PhiX genome), and raw error rate at the read length of 50. The results are shown in FIGS. 1 and 2, and in Table 1. As the results demonstrated, addition of betaine to the nucleotide incorporation mixes significantly improves sequencing performance on GeneReader, reducing the error rate at read-length 50 by more than 50%.

TABLE 1

| | Control | With Betaine |
|---|---|---|
| Library mapped reads best tile 50 cycles 1k/1k | 4891 | 6968 |
| Library perfectreads best tile 50 cycles 1k/1k | 1309 | 3673 |
| Library % perfect best tile 50 cycles 1k/1k | 26.76 | 52.71 |
| Library error best tile 50 cycles 1k/1k, % | 5.45 | 2.63 |

1. A method for sequencing a nucleic acid by detecting the identity of a nucleotide analogue incorporated into primer extension strand in a polymerase reaction, comprising:
a) providing i) an additive that eases secondary structure of DNA; ii) sequencing primer; (iii) single-stranded template attached to a solid surface; (iv) polymerase and v) a mixture of different nucleotide analogues, wherein each different nucleotide analogue comprises a base selected from the group consisting of adenine, guanine, cytosine, thymine, and uracil, and their analogues; and a unique label attached through a cleavable linker to the base or to an analogue of the base; a deoxyribose; and a cleavable chemical group at the 3′-position of the deoxyribose;
b) hybridizing the sequencing primer to the template so as to create a primer-template complex;
c) adding said additive to said mixture of different nucleotide analogues so as to create an extension solution;
d) exposing said primer-template complex to said extension solution under conditions such that the sequencing primer is extended by incorporating a first nucleotide analogue therein with said polymerase in a polymerase reaction so as to create a primer extension strand, wherein the incorporated nucleotide analogue terminates the polymerase reaction; and
e) detecting the unique label attached to the nucleotide analogue that has been incorporated into the growing strand of DNA, so as to thereby identify the incorporated nucleotide analogue.

2. The method of claim 1, further comprising f) cleaving the cleavable linker between the nucleotide analogue that was incorporated into primer extension strand and the unique label; and cleaving the cleavable chemical group at the 3′-position of the deoxyribose to leave an —OH group.

3. The method of claim 2, further comprises g) extending the sequencing primer by incorporating a second nucleotide analogue therein.

4. The method of claim 3, further comprising h) detecting the unique label attached to said second nucleotide analogue so as to thereby identity of the second incorporated nucleotide.

5. The method of claim 1, wherein said additive is betaine.

6. A method for sequencing a nucleic acid by detecting the identity of a nucleotide analogue incorporated into primer extension strand in a polymerase reaction, comprising:
a) providing i) sequencing primer, said sequencing primer hybridized to (ii) single-stranded template attached to a solid surface; (iii) polymerase and iv) an extension solution comprising an additive that eases secondary structure of DNA and one or more different nucleotide analogues, wherein each different nucleotide analogue comprises a base selected from the group consisting of adenine, guanine, cytosine, thymine, and uracil, and their analogues; and a unique label attached through a cleavable linker to the base or to an analogue of the base; a deoxyribose; and a cleavable chemical group at the 3′-position of the deoxyribose;
b) exposing said primer hybridized to said template to said extension solution and said polymerase under conditions such that the sequencing primer is extended by incorporating a first nucleotide analogue therein with said polymerase in a polymerase reaction so as to create a primer extension strand, wherein the incorporated nucleotide analogue terminates the polymerase reaction; and
c) detecting the unique label attached to the nucleotide analogue that has been incorporated into the growing strand of DNA, so as to thereby identify the incorporated nucleotide analogue.

7. The method of claim 6, further comprising d) cleaving the cleavable linker between the nucleotide analogue that was incorporated into primer extension strand and the unique label; and cleaving the cleavable chemical group at the 3′-position of the deoxyribose to leave an —OH group.

8. The method of claim 7, further comprising e) extending the sequencing primer by incorporating a second nucleotide analogue therein.

9. The method of claim 8, further comprising f) detecting the unique label attached to said second nucleotide analogue so as to thereby identity of the second incorporated nucleotide.

10. The method of claim 6, wherein said additive is betaine.
